(19) United States
(12) Patent Application Publication
(10) Pub. No.: US 2023/0062081 A1
(43) Pub. Date: Mar. 2, 2023

(54) SYSTEMS AND METHODS FOR PROVOKING AND MONITORING NEUROLOGICAL EVENTS

(71) Applicant: Eysz Inc., Piedmont, CA (US)

(72) Inventors: Rachel Kuperman, Piedmont, CA (US); Parth Amin, Cary, NC (US); Julia Martinez Franks, Piedmont, CA (US); Jordan DeLong, Piedmont, CA (US)

(21) Appl. No.: 17/823,400

(22) Filed: Aug. 30, 2022

Related U.S. Application Data
(60) Provisional application No. 63/239,158, filed on Aug. 31, 2021.
(60) Provisional application No. 63/243,896, filed on Sep. 14, 2021.

(51) Int. Cl. A61B 5/00; A61B 5/08; A61B 7/00; G16H 40/67

(52) U.S. Cl. CPC A61B 5/4094; A61B 5/0816; A61B 7/003; G16H 40/67; A61B 5/743; A61B 5/744

(57) ABSTRACT

Systems and methods for provoking and monitoring neurological events are described herein. In some embodiments, a method for monitoring a patient includes outputting a user interface configured to guide the patient in performing a provocation sequence for a neurological event. The method can also include obtaining patient data indicative of a state of the patient during the provocation sequence. The method can further include evaluating suitability of the patient data for detecting an occurrence of the neurological event. The method can include outputting feedback via the user interface based on the evaluation.

### CROSS-REFERENCE TO RELATED APPLICATION(S)

The present application claims the benefit of priority to U.S. Provisional Application No. 63/239,158, filed Aug. 31, 2021, and U.S. Provisional Application No. 63/243,896, filed Sep. 14, 2021, each of which is incorporated by reference herein in its entirety.

### TECHNICAL FIELD

The present technology generally relates to personalized healthcare, and in particular, to systems and methods for provoking and monitoring neurological events.

### BACKGROUND

Epilepsy is a neurological condition characterized by recurrent seizures. It is one of the oldest recorded diseases in the world, dating back several thousand years. Childhood absence epilepsy (CAE), an epilepsy syndrome with absence seizures, is the most common pediatric epilepsy syndrome, affecting 10-17% of all children with epilepsy. However, absence seizures are notoriously difficult to detect using conventional techniques. Absence seizures typically occur many times daily and involve brief staring spells that last for a few seconds, with immediate return to the baseline level of awareness and activity. As loss of consciousness may occur at any time and without warning, absence seizures can have a significant impact on quality of life. Accidental injury is common, with 20% of young adults—or 3% per year—reportedly suffering an injury during an absence seizure. The clinical course of CAE is variable, and remission rates are far lower than in other idiopathic epilepsies. For example, in five prospective cohort studies, the proportion of seizure-free subjects was only 57%-74%. Up to 25% of CAE participants may have cognitive or language impairments, and more than 50% have psychiatric diagnoses when formally assessed. The result of this burden is that children with CAE are more likely to require special education classes and repeat a grade, less likely to graduate high school, and more likely to be underemployed.

The goal of epilepsy treatment is to stop or reduce seizures with minimal side effects as quickly as possible. Access to reliable seizure data is fundamental to providing optimal epilepsy care from diagnosis to treatment. However, more than 99% of seizures occur outside of clinical settings. Patient self-reporting is the standard technique for measuring seizure data outside of the hospital, but studies have shown that self-reporting data is typically less than 50% accurate. The reliance on poor data contributes to the following challenges in epilepsy diagnosis and management: (1) it typically takes up to 7 years to make a diagnosis of epilepsy, (2) the misdiagnosis rate can be as high as 30%, (3) there is minimal data to help clinicians decide which drug (out of more than 20 currently available medications) to use for initial treatment, (4) the conventional trial and error approach in choosing the type and dosage of medication may result in overdosing (e.g., excess side effects) or underdosing (e.g., ongoing seizure activity), (5) the use of additional tests to supplement the poor data during the treatment cycle may be costly and ineffective, and (6) it takes on average 18 years to refer a patient with refractory epilepsy to an epilepsy center to be evaluated for epilepsy surgery, which may have a much higher success rate for seizure freedom in medication refractory patients than medications alone. Accordingly, improved systems and methods for monitoring and managing epilepsy, and/or other neurological diseases and conditions, are needed.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a schematic diagram of a computing environment in which a system for monitoring and managing neurological diseases and conditions can operate, in accordance with embodiments of the present technology.

FIG. 2 is a flow diagram illustrating a method for monitoring a patient, in accordance with embodiments of the present technology.

FIG. 3 is a flow diagram illustrating a provocation sequence, in accordance with embodiments of the present technology.

FIG. 4 is a flow diagram illustrating a method for monitoring a patient's breathing, in accordance with embodiments of the present technology.

FIG. 5 is a flow diagram illustrating a method for monitoring a patient's face, in accordance with embodiments of the present technology.

FIG. 6A illustrates a user interface for displaying guidance for preparing for a provocation attempt, in accordance with embodiments of the present technology.

FIG. 6B illustrates a user interface for displaying guidance for positioning and recording a patient's face, in accordance with embodiments of the present technology.

FIG. 6C illustrates a user interface for displaying guidance for performing a hyperventilation sequence, in accordance with embodiments of the present technology.

FIG. 6D illustrates a user interface for submitting self-reporting data, in accordance with embodiments of the present technology.

FIG. 7 is a flow diagram illustrating a method for detecting a neurological event, in accordance with embodiments of the present technology.

### DETAILED DESCRIPTION

The present technology relates to systems and methods for monitoring and/or provoking neurological events in a patient. Currently, healthcare providers have no accurate and convenient way to identify changes in seizure frequency and/or severity between office visits. Voluntary provocation of seizures using techniques such as hyperventilation may aid in diagnosing and monitoring certain epilepsies such as absence epilepsy. However, conventional approaches for performing voluntary hyperventilation lack standardization and are not integrated into clinical workflows. Additionally, conventional approaches for patient monitoring during seizure provocation may not be suitable for use in outpatient settings, such as the patient's home. Video electroencephalography (VEEG) is the gold standard for identifying the occurrence of an absence seizure in a patient, but is typically only available in hospitals, such that accessibility, lack of trained professionals, and costs are barriers to widespread adoption. Additionally, VEEG is impractical for frequent seizure monitoring and only provides information while in use. Patient self-reporting data is conventionally used to determine seizure occurrences outside of healthcare settings, but self-reporting data is frequently inaccurate, which can have a substantial impact on outpatient management and treatment. Patients frequently struggle to report on seizure counts, particularly in absence epilepsy, given the brief nature of the seizures and the subtle clinical signs.

To address these and other challenges, the present technology provides techniques for patient monitoring that can be consistently and reliably implemented in outpatient settings. In some embodiments, for example, a method for monitoring a patient includes outputting a user interface configured to guide the patient in performing a provocation sequence for a neurological event, such as an absence seizure, other seizure type, or other neurological symptom (e.g., syncope). The method can also include obtaining patient data indicative of a state of the patient during the provocation sequence. For example, the patient data can include eye tracking data, facial tracking data, and/or other data types suitable for detecting the neurological condition of the patient. In some embodiments, the patient data is obtained using sensors (e.g., camera, microphone) built into or connected to a patient's smartphone, tablet, laptop, or other computing device. The method can further include evaluating suitability of the patient data for detecting an occurrence of the neurological event. In some embodiments, for instance, the method can include determining whether the patient's breathing rate during the sequence is within a targeted range to produce a hyperventilation-induced seizure. As another example, the method can include whether the patient's face and/or eyes are sufficiently visible in a video recording to be used for seizure detection. The method can include outputting feedback via the user interface based on the evaluation, such as instructions to increase or decrease breathing rate, reposition the patient's face relative to the camera, etc.

The present technology can provide many benefits for monitoring and managing neurological diseases and conditions. For example, the systems, methods, and devices described herein can provide guidance to assist a patient in voluntarily provoking a neurological event (e.g., a seizure), including real-time feedback, thus allowing provocation to be performed in a consistent, standardized manner, without requiring assistance from trained healthcare professionals or the use of specialized equipment, thus improving the accessibility of patient care. Additionally, the present technology can generate objective, reproducible data of the patient's state during the provocation attempt, thus providing additional information regarding the current disease burden that may be used to supplement or replace self-reporting data. This approach can improve disease management, e.g., by assisting physicians in medical decision making, such as deciding when and how to perform medication titrations and/or other adjustments to the patient's therapeutic regimen, deciding when to perform an EEG to confirm seizure freedom, etc.

The techniques described herein can also be applied to other types of neurological diseases or conditions, such as disorders causing syncope or other loss of consciousness. For example, syncope is caused by a reduction in cerebral blood flow, resulting in loss of postural tone followed by spontaneous recovery. Although most etiologies of orthostatic syncope carry a benign prognosis, up to one-third of affected individuals experience recurrent and severe episodes, and syncope can be associated with significant impairments in quality of life. Provocative maneuvers such as changes in position can be used to identify the prodromal symptoms of syncope (e.g., pre-syncope) through eye movements (e.g., changes in pupillary tone, loss of spontaneous saccades) suggesting dysregulation in the autonomic nervous system that place the patient at risk for syncope. These symptoms can be monitored over time to determine if treatment for the underlying etiology has decreased the risk of syncope.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The headings provided herein are for convenience only and do not interpret the scope or meaning of the claimed present technology. Embodiments under any one heading may be used in conjunction with embodiments under any other heading.

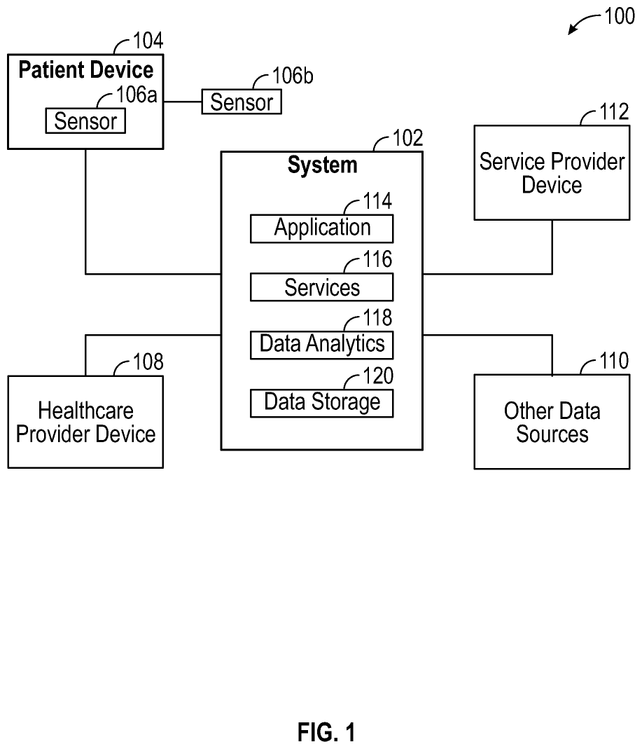

FIG. 1 is a schematic diagram of a computing environment 100 in which a system for monitoring and managing neurological diseases and conditions (“system 102”) can operate, in accordance with embodiments of the present technology. The system 102 is configured to monitor a patient who has been diagnosed with or is suspected of having a neurological disease or condition, such as epilepsy (e.g., focal epilepsy, generalized epilepsy, combined generalized and focal epilepsy, unknown epilepsy) or a disorder in which epilepsy is a symptom (e.g., Rett syndrome, tuberous sclerosis). In some embodiments, the system 102 is configured to receive and process various types of patient data, in order to generate diagnoses, analyses, reports, patient histories, treatment recommendations, etc., for the patient. For example, the system 102 can analyze patient data (e.g., eye tracking data, facial tracking data, audio data, EEG data) to determine whether the patient has experienced a neurological event (e.g., a seizure, loss of consciousness), as described in greater detail below.

The patient data can include any data relevant to the patient's health and/or treatment of a neurological disease or condition. For example, the patient data can include any of the following: eye tracking data, facial tracking data, tracking data for other body parts (e.g., head, arms, hands, legs, feet, torso), motion data (e.g., whether the patient is moving, stationary, lying down), cardiovascular data (e.g., heart rate, blood pressure, blood oxygenation levels, electrocardiogram (ECG) data), breathing data (e.g., breathing rate, breathing volume), body temperature data (e.g., skin temperature), brain monitoring data (e.g., EEG data), EMG data, electrodermal data, biomarker levels in body fluids (e.g., sweat, blood, tears), neuropsychological assessment data, self-reporting data (e.g., seizure diaries), treatment plan data (e.g., medications, dosages, neurostimulation parameters), dietary data, electronic health records, demographic data, medical history, familial medical history, test results, imaging data (e.g., MRI data), genetic data, and/or any other suitable data type.

In some embodiments, the system 102 is operably coupled to at least one patient device 104. The patient device 104 can be or include any suitable device capable of obtaining patient data. For example, the patient device 104 can be or include at least one sensor 106a configured to generate sensor data indicative of the patient's health state, and the sensor data can be processed (e.g., by the patient device 104 and/or system 102) to determine the patient data. Alternatively or in combination, the patient device 104 can be operably coupled to at least one separate sensor 106b. For example, the patient device 104 can receive sensor data from at least one separate sensor 106b via wired or wireless communication techniques (e.g., Bluetooth, WiFi, USB). Although FIG. 1 illustrates a single onboard sensor 106a and a single separate sensor 106b, in other embodiments, the patient device 104 can include and/or be coupled to any suitable number of sensors, such as two, three, four, five, or more onboard sensors 106a and/or two, three, four, five or more separate sensors 106b.

Examples of sensor types suitable for use with the present technology include, but are not limited to: eye tracking devices (e.g., electrooculography (EOG)-based devices, video-based devices, laser-based devices, LIDAR-based devices), facial tracking devices (e.g., EMG-based devices, video-based devices), breath rate monitors, brain monitoring devices (e.g., EEG sensors), motion sensors (e.g., accelerometers, gyroscopes), position sensors, orientation sensors, haptic sensors, pressure sensors, flow rate sensors, activity sensors, imaging devices (e.g., cameras), audio sensors (e.g., microphones), heart rate sensors, blood pressure sensors, pulse oximeters, temperature sensors, ECG sensors, EMG sensors, electrodermal sensors, and electrochemical sensors. Optionally, a single device can include multiple sensor types. For example, a single device can include sensors for eye tracking, facial tracking, and/or brain monitoring. As another example, a single device can include an imaging device and an audio sensor.

For example, an eye tracking device can be any type of device configured to produce eye tracking data characterizing the movement, shape, and/or other relevant characteristics of one or both of the patient's eyes. The eye tracking data can include any of the following types of data: blink rate, blink duration, eye eccentricity, eye gaze angle, pupil size, pupil constriction amount, pupil constriction velocity, pupil dilation amount, pupil dilation velocity, pupil location, pupil rotation, pupil area to iris area ratio, hippus, eyelid movement rate, eyelid openings, eyelid closures, eyelid height, upward eyeball movements, downward eyeball movements, lateral eyeball movements, relative eye movements, eye rolling, jerky eye movements, saccadic velocity, saccadic direction, torsional velocity, torsional direction, gaze direction, gaze scanning patterns, or eye activity during sleep. The eye tracking device can use various types of sensor technologies, including, but not limited to, EOG, video, laser, and LIDAR.

As another example, a facial tracking device can be any type of device configured to produce facial movement data characterizing the motion, shape, and/or other relevant features of the patient's face. For example, the facial movement data can include measurements of and/or changes in any of the following: distance between the patient's eyes, distance between the patient's eyelids, width of the patient's nose, center of the patient's nose, depth of the patient's eye sockets, shape of the patient's cheekbones, length of the patient's jawline, distance between the patient's mouth edges center of the patient's mouth, or focal weakness. The facial tracking device can use various types of sensor technologies, such as video, movement sensors, EOG, and/or EMG.

In a further example, a brain monitoring device can be any type of device configured to produce brain monitoring data characterizing the activity of the patient's brain. For example, the brain monitoring device can be an EEG device that includes a plurality of electrodes configured to measure electrical activity of various parts of the brain.

The patient device 104 and/or sensors 106a, 106b can have any suitable form factor, such as wearable devices (e.g., fitness trackers, smartwatches, smart glasses, smart contact lenses, VR/AR headsets), implantable devices, non-implantable devices, mobile devices, fixed devices (e.g., devices that are attached to a wheelchair or other object), etc. In some embodiments, for example, the patient device 104 is a computing device, such as a mobile device (e.g., smartphone, tablet), laptop, personal computer, etc. The computing device can include software and hardware components (e.g., processors, memory) configured to perform the various operations described herein. As described above, the computing device can include one or more onboard sensors 106a (e.g., a built-in camera, a built-in microphone) and/or can be operably coupled to one or more separate sensors 106b (e.g., an eye tracking device and/or facial tracking device, web camera, microphone). The computing device can be configured to implement a software application (e.g., a web-based application and/or a mobile application) that performs various data collection and/or analysis operations, as described further below.

Optionally, the patient device 104 can include user interface functionality that allows a patient (or another user) to input data into the patient device 104 and/or view data output by the patient device 104. For example, the patient device 104 can include or be operably coupled to input tools (e.g., a touchscreen, keyboard, mouse, joystick, microphone) that allow a user to enter patient data (e.g., self-reporting data such as seizure diaries, demographic data, medication data, patient history, contact information, responses to questionnaires). The patient device 104 can also include a display that outputs information to the user, such as patient data, sensor data, reports, feedback, treatment recommendations, instructions for performing active tasks (e.g., reading, watching a video, stimuli-based tasks), etc. In some embodiments, for example, the display is configured to output a user interface providing guidance for provoking a neurological event, as described in greater detail below.

The monitoring performed by the patient device 104 can be performed during daytime, nighttime, while the patient is awake, while the patient is sleeping, while the patient is resting, while the patient is engaging in activities, and/or any other suitable time frame. The monitoring can be performed at any suitable frequency, such as once every 30 seconds, 1 minute, 2 minutes, 5 minutes, 10 minutes, 30 minutes, 1 hour, etc. Optionally, the patient device 104 can prompt the patient to initiate monitoring at specified time intervals.

The patient device 104 can store and process the received patient data, and/or can transmit the patient data to the system 102 for storage and processing. For instance, the patient device 104 can process sensor data received from one or more sensors before transmitting to the system 102, or can transmit the raw sensor data to the system 102 without processing. The patient device 104 can transmit the data to the system 102 continuously, at predetermined intervals (e.g., once per day, week, month), when new or updated data is available, when the system 102 sends a request to the patient device 104, or any other suitable manner. Any of the operations described herein as being performed locally on the patient device 104 can alternatively or additionally be performed remotely on the system 102, and vice-versa.

In some embodiments, the system 102 is also operably coupled to at least one healthcare provider device 108. The healthcare provider device 108 can be a computing system or device (e.g., a mobile device, laptop, personal computer, server) that is associated with a healthcare professional providing care for the patient, such as a primary care physician, neurologist, etc. For example, the healthcare professional can generate patient data such as electronic health records, neuropsychological assessments, test results, diagnoses, treatment plans, etc., that are stored at the healthcare provider device 108. The healthcare provider device 108 can transmit the patient data to the system 102 continuously, at predetermined intervals (e.g., once per day, week, month), when new or updated data is available, when the system 102 sends a request to the healthcare provider device 108, or any other suitable manner.

Optionally, the system 102 can be operably coupled one or more other data sources 110 that provide additional patient data. For example, the other data sources 110 can include electronic health records, electronic data capture systems, publicly accessible databases (e.g., regulatory databases, research databases, academic databases), and/or any other external source of data relevant to the patient's health. In some embodiments, the system 102 communicates with the other data sources 110 using suitable protocols (e.g., Fast Healthcare Interoperability Resources (FHIR)) and/or application programming interfaces (APIs).

The system 102 can be configured to process and analyze the patient data received from the patient device 104, healthcare provider device 108, and/or other data sources 110 to monitor and/or manage the patient's neurological disease or condition. For example, based on the patient data, the system 102 can determine the patient's past, current, and/or predicted future health state, such as whether the patient has experienced, is experiencing, and/or is likely to experience a neurological event such as a seizure. As another example, the system 102 can monitor the patient during periods between neurological events (e.g., interictal periods between seizures) to assess the health state (e.g., neurocognitive state, encephalopathy) of the patient while the patient is exhibiting relatively normal function. In some embodiments, the system 102 implements one or more machine learning algorithms that are trained to analyze the patient data to detect whether any neurological events occurred during the monitoring period, as described elsewhere herein. Additional details of the data processing and analysis operations that can be performed by the system 102 are provided below.

Optionally, the system 102 can generate a report for the patient including various types of information to assist a healthcare provider and/or the patient in managing the patient's disease or condition. For example, the report can include information regarding the history and progression of the patient's disease or condition over time. The report can also include a personalized, patient-specific dose-response profile that assesses how the patient responds to various treatments, in terms of therapeutic efficacy (e.g., seizure control) as well as toxicity (e.g., side effects). Optionally, the report can include recommendations for the patient's treatment that are predicted to improve efficacy and/or reduce toxicity. Additional details of methods for determining the dose-response profile and/or other personalized treatment recommendations are provided in U.S. Patent Application Publication No. 2022/0180993, which is incorporated by reference herein in its entirety.

Optionally, the system 102 can be operably coupled to at least one service provider device 112. The service provider device 112 can be a computing system or device (e.g., a personal computer, workstation) that is associated with a service provider (e.g., a healthcare professional such as a neurologist or technician) who is affiliated with the system 102. For example, the service provider can review the patient data received by the system 102 to confirm whether the data is suitable for subsequent analysis and data processing operations. As another example, the service provider can annotate or otherwise modify the patient data (e.g., label occurrences of neurological events) to facilitate subsequent analysis performed by the system 102. Optionally, the service provider can review the analysis results (e.g., reports) produced by the system 102 to confirm accuracy, provide feedback, make modifications, add comments, etc. Accordingly, any of the operations described herein as being performed automatically by the system 102 can alternatively be performed manually by the service provider, or can be performed in a semi-automated manner (e.g., the system 102 automatically produces results for manual review by the service provider). In other embodiments, however, the service provider is optional and the service provider device 112 can be omitted.

In some embodiments, the system 102 transmits the analysis results (e.g., the report) to the healthcare provider device 108 so the healthcare provider can evaluate the patient's condition, and, if appropriate, adjust a prescribed treatment regimen. The results can be transmitted at any suitable time interval, such as daily, weekly, bi-weekly, when updated data is available, in response to a request from the healthcare provider device 108, etc. Optionally, after reviewing the results, the healthcare provider can provide feedback to the system 102 via the healthcare provider device 108. For example, if the healthcare provider determines that the system 102 has incorrectly identified certain neurological events, the healthcare provider can send this feedback to the system 102 so the system 102 can update its analysis and generate a revised report. As another example, if the healthcare provider determines that a treatment recommended by the system 102 is not appropriate for the particular patient, the system 102 can update its treatment recommendation algorithm so the treatment is not recommended for that patient (and/or for similar patients) in the future.

The system 102 shown in FIG. 1 can be configured in many different ways. For example, the system 102 can be or include one or more computing systems or devices having software and hardware components (e.g., processors, memory) configured to perform the various operations described herein. In some embodiments, the system 102 is implemented as a distributed cloud platform across any suitable combination of hardware and/or virtual computing resources.

Optionally, the system 102 can include a plurality of functional components, such as an application layer 114, a services layer 116, a data analytics layer 118, and/or data storage 120. The application layer 114 can be configured to generate external user interfaces (e.g., mobile interfaces, web interfaces) that are displayed on the patient device 104, healthcare provider device 108, and/or service provider device 112. For example, patients can interact with the system 102 via a mobile interface on a mobile application running on the patient device 104, while healthcare providers and service providers can interact with the system 102 via a web interface of a web-based application accessed via the healthcare provider device 108 and service provider device 112, respectively. In some embodiments, the system 102 implements a web-based application that allows users (e.g., patients, healthcare providers, service providers) to perform various actions depending on their role, such as reviewing and/or annotating patient data, reviewing reports, performing administrative tasks (e.g., registering patients), and/or updating settings on the mobile application. The application layer 114 can also communicate data to and from the patient device 104, healthcare provider device 108, and/or service provider device 112, such as patient data, analysis results, etc.

The services layer 116 can manage the communications and/or interfaces to the patient device 104, healthcare provider device 108, service provider device 112, and/or other data sources 110. For example, the services layer 116 can pre-process incoming data (e.g., patient data, clinical data, sensor data such as video data) and outgoing data (e.g., reports), and link the data to the appropriate devices. Optionally, the services layer 116 can control the collection, usage, and/or transfer of data in compliance with applicable privacy and security regulations (e.g., HIPAA). For example, the services layer 116 can ensure that patient data and/or other sensitive data is transmitted with appropriate encryption protocols for security purposes.

The data analytics layer 118 can analyze incoming data and prepare data for output (e.g., as reports). In some embodiments, the data analytics layer 118 analyzes patient data using statistical analysis, pattern recognition, machine learning, and/or other techniques to perform various operations, such as detecting recognizable patterns consistent with seizure events and generating seizure data indicative of the patient's seizure burden, including timestamps of seizure events, number of seizure events, duration of seizures, etc. The data analytics layer 118 can also generate electronic reports summarizing the results of the analysis, as described elsewhere herein. The data storage 120 can manage relationships between data and/or store data (e.g., patient data, analysis results) using database technologies known to those of skill in the art.

The various components 102-120 illustrated in FIG. 1 can communicate with each other via one or more communications networks, such as any of the following: a wired network, a wireless network, a metropolitan area network (MAN), a local area network (LAN), a wide area network (WAN), a virtual local area network (VLAN), an internet, an extranet, an intranet, and/or any other suitable type of network or combinations thereof. Alternatively or in combination, the components 102-120 can interact through any suitable wired or wireless communication technique, such as Bluetooth, WiFi, USB, etc. Additionally, in some embodiments, the patient device 104, healthcare provider device 108, and service provider device 112 can communicate with each other directly using any suitable communication technique, rather than via the system 102 as shown in FIG. 1.

Although FIG. 1 illustrates a single patient device 104 that is used to collect data from a single patient, in other embodiments, the system 102 can be operably coupled to multiple patient devices 104, each patient device 104 being associated with a different patient. Accordingly, the system 102 can be configured to receive and analyze patient data from a large number of patients, such as tens, hundreds, thousands, or tens of thousands of patients. In such embodiments, the data storage 120 can serve as a centralized repository for patient data from multiple patients. Optionally, the system 102 can analyze the patient data from multiple patients to generate population-level statistics, identify patterns and/or trends across large numbers of patients, use data from other patients to generate dose-response profiles, predictions, and/or treatment recommendations for a particular patient, etc. In some embodiments, data from multiple patients is used to create training data for the machine learning algorithms described herein.

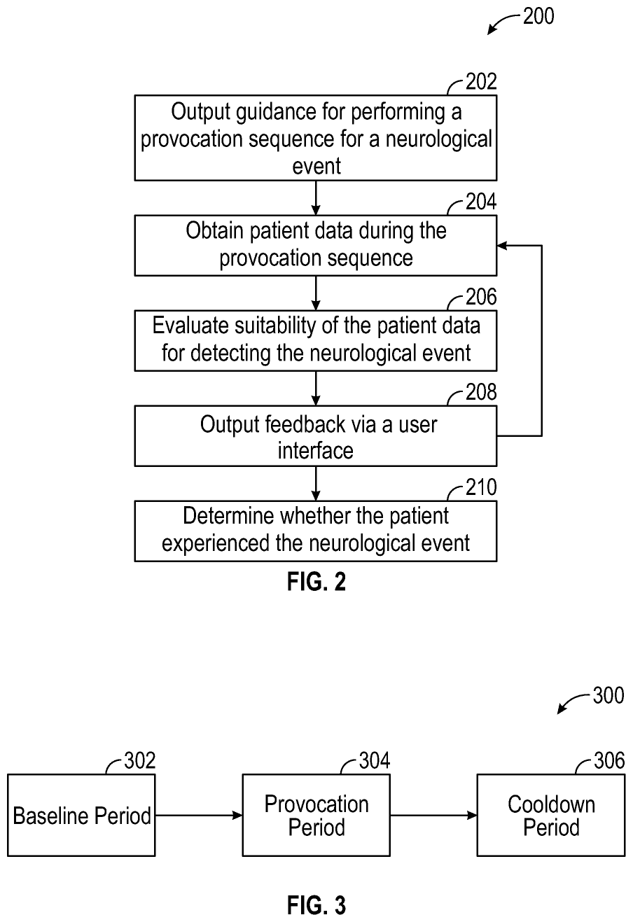

FIG. 2 is a flow diagram illustrating a method 200 for monitoring a patient, in accordance with embodiments of the present technology. The method 200 can be performed by any embodiment of the systems and devices described herein, such as by a computing system or device including one or more processors and a memory storing instructions that, when executed by the one or more processors, cause the computing system or the device to perform some or all of the operations described herein. For example, some or all of the processes of the method 200 can be performed by the patient device 104 (e.g., a mobile device or other computing device). Alternatively or in combination, some or all of the processes of the method 200 can be performed by the system 102 of FIG. 1 (e.g., a cloud-based software platform).

The method 200 begins at block 202 with outputting guidance for performing a provocation sequence for a neurological event. The neurological event can be a symptom associated with the neurological disease or condition of the patient, and can be any pathological event that can be induced in the patient via certain voluntary actions. For example, in some embodiments, the neurological disease or condition is epilepsy (or a disorder in which epilepsy is a symptom) and the neurological event is a seizure, such as an absence seizure, atypical absence seizure, tonic-clonic seizure, clonic seizure, tonic seizure, atonic seizure, myoclonic seizure, simple partial seizure, complex partial seizure, secondary generalized seizure, and/or infantile spasm. As another example, the neurological event can be a loss of consciousness, such as syncope. Examples of syncopal/presyncopal disorders include vasovagal syncope (VVS), postural orthostatic tachycardia syndrome (POTS), orthostatic hypotension (OH,) and carotid sinus hypersensitivity (CSH).

The provocation sequence of block 202 can include one or more patient actions that are likely to induce the neurological event in the patient (also referred to herein as “provocation actions”). For example, certain types of seizures can be provoked through techniques such as photic simulation, auditory stimulation, hyperventilation, visual patterns, reading, and/or concentration. As another example, syncope or pre-syncope can be provoked by postural changes; prolonged standing; painful, frightening, and/or disturbing stimuli; emotional distress; instrumentation; and/or breathing (e.g., hyperventilation).

In some embodiments, the provocation sequence is configured to induce a neurological event (e.g., a seizure, syncope) in the patient via hyperventilation. Voluntary hyperventilation can be used to assist in diagnosing and monitoring certain epilepsies such as absence epilepsy (e.g, CAE). The mechanism by which voluntary hyperventilation and the resulting respiratory alkalosis induces absence seizures is likely related to cerebral blood vessel constriction, as well as altering the thalamocortical circuit elements responsible for production of epileptiform activity. The use of hyperventilation to definitively and quickly diagnose patients with absence epilepsy can eliminate the need for more burdensome protocols, such as an extended EEG recording procedure to capture spontaneous seizures. Additionally, hyperventilation can be performed in an outpatient setting to provide reliable information regarding the patient's disease burden and/or response to treatment.

In some embodiments, the provocation sequence includes one or more of the following provocation actions: breathing at a particular rate (e.g., from 20 to 24 breaths per minute), viewing a visual stimulus (e.g., graphics such as flickering lights, visual patterns, video clips), listening to an auditory stimulus (e.g., sounds, music, voices), reading text, changing position (e.g., lying down to sitting, sitting to standing), and/or focusing on a particular location and/or object. The timing for each provocation action can be varied as desired. For example, a provocation action can be performed for any suitable duration, such as a duration within a range from 1 minute to 20 minutes, 1 minute to 10 minutes, 1 minutes to 5 minutes, or 5 minutes to 10 minutes. In some embodiments, a provocation action is performed for a duration of at least 1 minute, 2 minutes, 3 minutes, 4 minutes, 5 minutes, 10 minutes, 15 minutes, or 20 minutes. In embodiments where the provocation sequence includes performing multiple provocation actions, the provocation actions can be performed sequentially or concurrently.

Optionally, the provocation sequence can include one or more patient actions that are configured to place the patient in a baseline state (e.g., relatively normal neurological condition) in which the patient is not likely to experience any neurological events (also referred to herein as “neutral actions”). For example, it may be advantageous to have the patient be in a baseline state before the provocation attempt, e.g., to prepare the patient for provocation and/or to collect data characterizing the patient's baseline state. As another example, it may be desirable to guide the patient in returning to the baseline state after the provocation attempt, e.g., so the patient can recover. Examples of neutral actions include, but are not limited to: breathing at a normal rate (e.g., from 12 to 16 breaths per minute), viewing a neutral graphic (e.g., a video clip), and listening to a neutral audio signal.

The timing for each neutral action can be varied as desired. For example, a neutral action can be performed for any suitable duration, such as a duration within a range from 1 minute to 20 minutes, 1 minute to 10 minutes, 1 minutes to 5 minutes, or 5 minutes to 10 minutes. In some embodiments, a neutral action is performed for a duration of at least 1 minute, 2 minutes, 3 minutes, 4 minutes, 5 minutes, 10 minutes, 15 minutes, or 20 minutes. Optionally, in embodiments where the provocation sequence includes performing multiple neutral actions, the neutral actions can be performed sequentially or concurrently. In other embodiments, however, the provocation sequence may not include any neutral actions.

FIG. 3 is a flow diagram illustrating a representative example of a provocation sequence 300 that can be used to induce a neurological event in a patient, in accordance with embodiments of the present technology. As shown in FIG. 3, the provocation sequence 300 can include multiple stages, such as a baseline period 302, a provocation period 304, and a cooldown period 306. During the baseline period 302, the patient can be instructed to perform one or more neutral actions in order to place the patient in a baseline state. For example, to prepare for hyperventilation, the patient can be instructed to breathe normally and to watch a short video clip. The baseline period 302 can have a duration of no more than 5 minutes, 4 minutes, 3 minutes, 2 minutes, or 1 minute.

During the provocation period 304, the patient can be instructed to perform one or more provocation actions in order to attempt to provoke the neurological event. For example, to induce a hyperventilation-triggered seizure, the patient can be instructed to breathe at an elevated breathing rate (e.g., within a range from 20 to 24 breaths per minute, such as 22 breaths per minute) for a set time period. The provocation period 304 can have a duration of no more than 10 minutes, 9 minutes, 8 minutes, 7 minutes, 6 minutes, 5 minutes, 4 minutes, 3 minutes, 2 minutes, or 1 minute.

During the cooldown period 306, the patient can be instructed to perform one or more neutral actions to return to the baseline state. For example, after hyperventilating, the patient can be instructed to breathe normally and to watch a short video clip. The cooldown period 306 can have a duration of no more than 5 minutes, 4 minutes, 3 minutes, 2 minutes, or 1 minute. The cooldown period 306 can involve the same actions and/or duration as the baseline period 302, or can involve different actions and/or duration.

The provocation sequence 300 shown in FIG. 3 can be varied in many ways. For example, in other embodiments, the baseline period 302 and/or cooldown period 306 can be omitted. Optionally, the provocation sequence 300 can include additional stages not shown in FIG. 3, such as additional baseline periods 302, provocation periods 304, and/or cooldown periods 306. For instance, the provocation sequence 300 can include multiple provocation periods 304 (e.g., two, three, four, five, or more provocation periods 304). In such embodiments, some or all of the provocation periods 304 can involve the same provocation actions and durations, or some or all of the provocation periods 304 can involve different provocation actions and/or durations. The provocation periods 304 can be interspersed with cooldown periods 306 and/or baseline periods 302 to allow the patient to recover between provocation attempts.

Referring again to FIG. 2, the guidance of block 202 can be configured to assist the patient in performing the appropriate actions and timing for the provocation sequence. Optionally, the guidance of block 202 can also be configured to ensure that the patient can be adequately monitored while performing the provocation sequence. As described below in connection with block 204, the patient can be monitored using at least one sensor configured to generate data indicating whether any neurological events occurred during provocation. In such embodiments, the patient may need to be in a particular position, wearing a particular device, performing particular actions, etc., in order for the sensor to obtain data of the patient that is suitable for subsequent analysis. For example, in embodiments where the sensor is an imaging device (e.g., a camera) configured to obtain image and/or video data of the patient's face, the guidance can include instructions for positioning the patient within the field of view of the imaging device. As another example, in embodiments where the sensor is a wearable eye tracking device, the guidance can include instructions on how to properly wear the eye tracking device. The guidance can also provide corrective feedback if the patient is not situated properly for monitoring, as described below in connection with block 208.

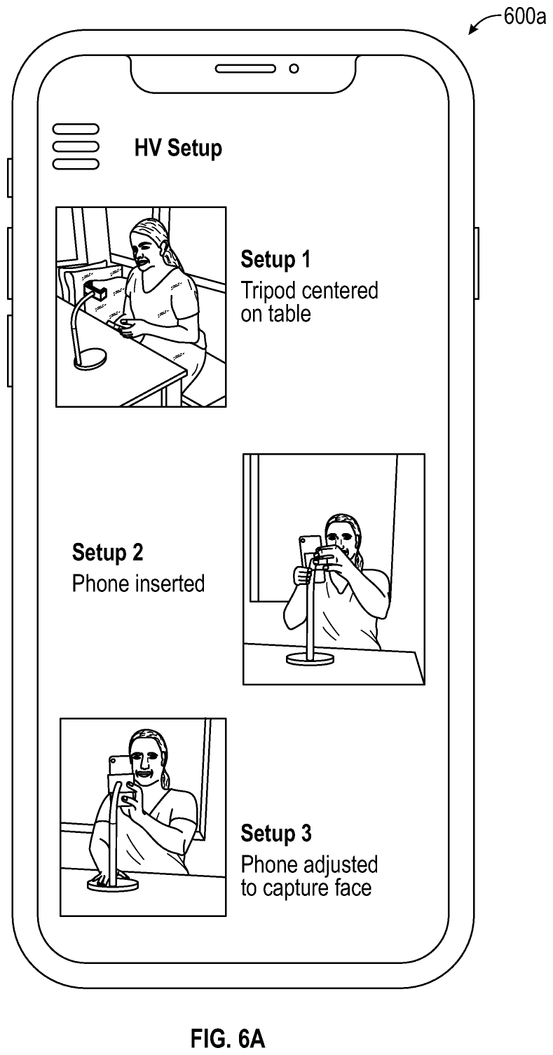

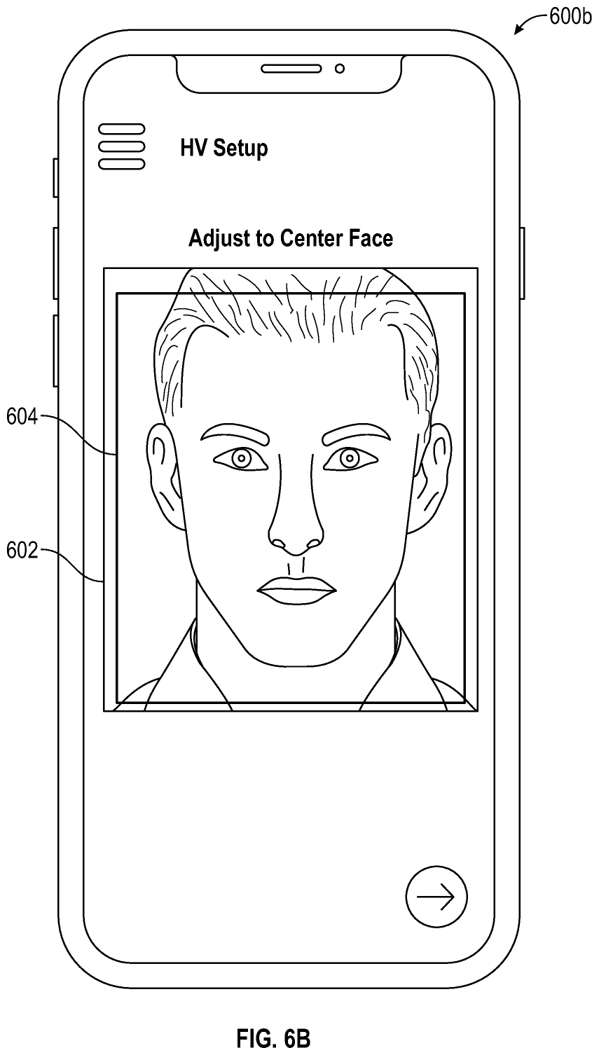

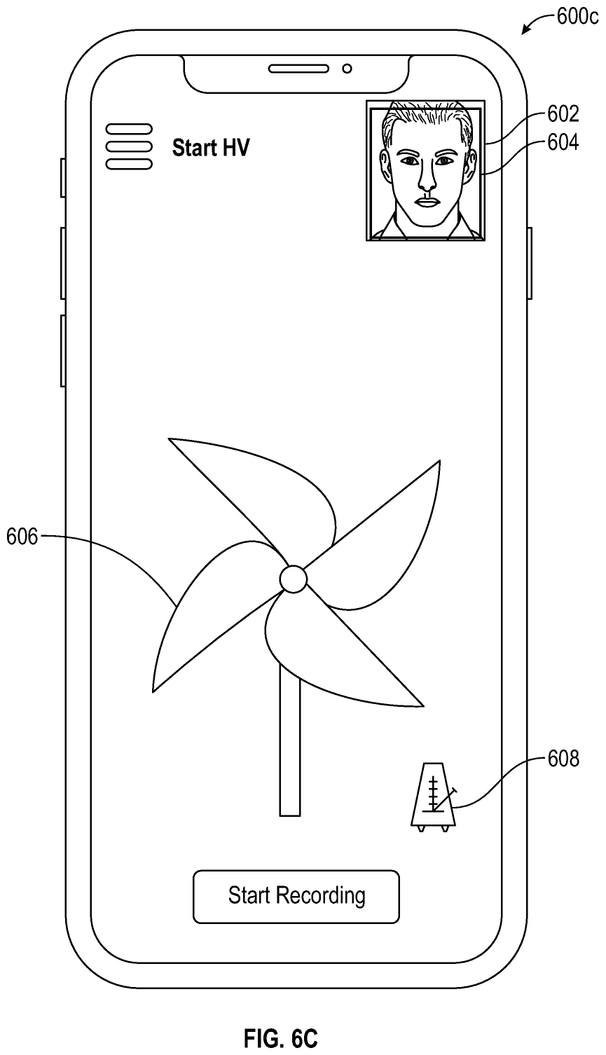

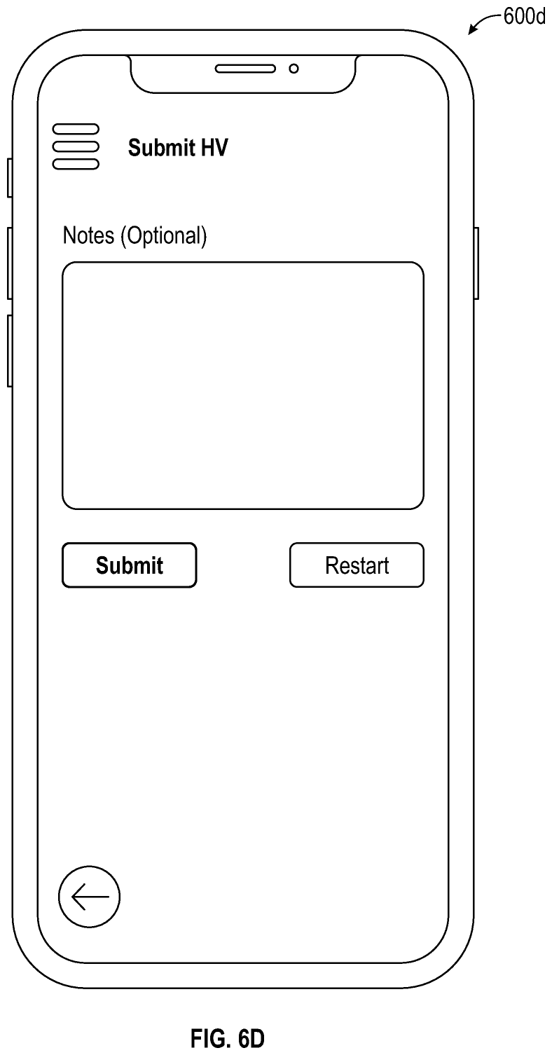

The guidance can be presented to the patient in many different ways. In some embodiments, the guidance is shown on a user interface of a software application (e.g., a mobile application or web application) running on a mobile or stationary computing device (e.g., smartphone, tablet, laptop, personal computer) of the patient. The guidance can include animations, graphics, videos, text, sounds, haptics, and/or any other output that can assist the patient in performing the provocation sequence and/or any other relevant tasks. For example, in embodiments where the provocation sequence involves hyperventilation, the guidance can include an animation (e.g., pinwheel, sailboat, candle, tissue) that moves, changes, or otherwise reflects the patient's breathing rate. Additional details of user interfaces providing guidance suitable for use with the method 200 are described below in connection with FIGS. 6A-6D.

At block 204, the method 200 can include obtaining patient data during the provocation sequence. The patient data can include any data type that can be used to determine: (1) whether the patient successfully provoked the neurological event, as well as the characteristics (e.g., timestamps, duration, severity, frequency) of any neurological events that occurred; (2) whether the provocation actions were properly performed; and/or (3) whether the patient was properly monitored during the provocation sequence. For example, the patient data can include eye tracking data, facial tracking data, tracking data for other body parts, motion data, cardiovascular data, breathing data, body temperature data, brain monitoring data, EMG data, electrodermal data, biomarker data, and so on.

In some embodiments, the patient data is obtained using at least one sensor (e.g., the sensor 106a and/or the sensor 106b of FIG. 1). The sensor can be any of the sensor types described herein, such as an eye tracking device, facial tracking device, breath rate monitor, brain monitoring device, motion sensor, orientation sensor, haptic sensor, pressure sensor, flow rate sensor, activity sensor, imaging device, audio sensor, heart rate sensor, blood pressure sensor, pulse oximeter, temperature sensor, electrophysiological sensor (e.g., EEG sensor, EOG sensor, ECG sensor, EMG sensor, electrodermal sensor), electrochemical sensor, etc. Any suitable number of sensors can be used, such as one, two, three, four, five, or more sensors.

For example, an audio sensor (e.g., a microphone) can be used to obtain audio data of the patient's breathing during the provocation sequence. In some embodiments, the audio data is used to measure the characteristics of the patient's breathing (e.g., breathing rate) to assess whether the patient is properly hyperventilating to provoke a seizure. As another example, an imaging device (e.g., a camera) can be used to obtain video data of a patient's eyes and/or face during the provocation sequence, and the video data can subsequently be analyzed to generate eye tracking data and/or facial tracking data indicative of the patient's neurological state (e.g., whether the patient experienced any seizures). Alternatively or in combination, eye tracking data (e.g., EOG data) and/or facial tracking data can be generated using a remote or wearable device, such as an eye tracking device, an electrophysiological recording device, a VR/AR headset including embedded sensors, etc.

Optionally, an EEG device can be used to obtain EEG data of the patient during the provocation sequence, as an alternative to or in combination with the audio sensor and/or the imaging device. In embodiments where EEG data is obtained in conjunction with video data, the process of block 204 can further include temporally synchronizing the EEG data with the video data. Thus, the video of the patient's eyes and/or face can be displayed together with the relevant EEG data during user review, as described further below in connection with block 210.

Referring again to FIG. 3, certain types of patient data may be obtained throughout the entire provocation sequence 300. For example, video data and/or audio data of the patient can be continuously recorded during the baseline period 302, provocation period 304, and cooldown period 306. This approach can ensure that patient is properly situated for monitoring during the baseline period 302, before the patient attempts to provoke the neurological event. Additionally, patient data collected during the baseline period 302 and/or cooldown period 306 can be compared to the patient data collected during the provocation period 304, e.g., to determine the patient's baseline state versus the patient's state while experiencing the neurological event. Optionally, certain types of patient data can be obtained only during certain portions of the provocation sequence 300, such as during the baseline period 302 only, during the provocation period 304 only, during the cooldown period 306 only, during the baseline period 302 and cooldown period 306 only, etc.

Referring again to FIG. 2, at block 206, the method 200 can include evaluating suitability of the patient data for detecting the neurological event. The evaluation can be based on at least the following criteria: (1) whether the patient was properly performing the provocation action(s) when the patient data was obtained, and (2) whether the patient data adequately captures the patient's state during the provocation sequence.

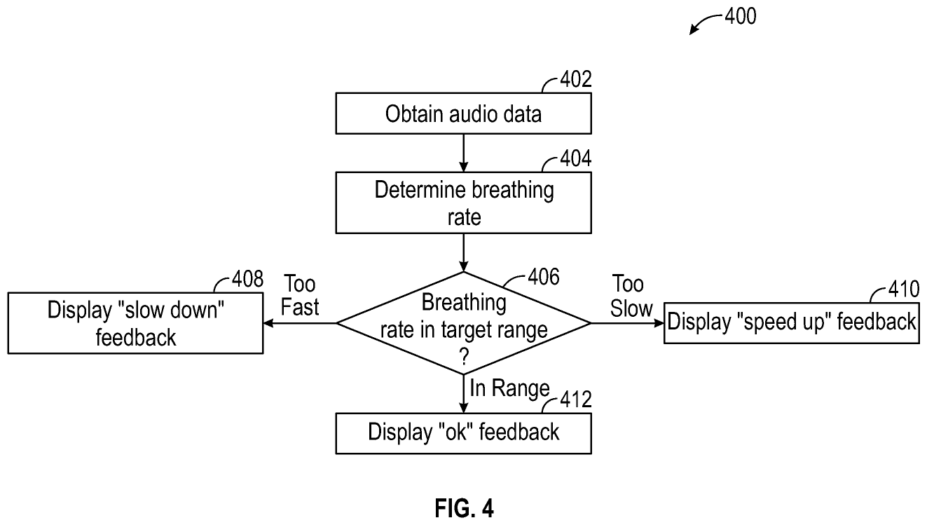

For example, in the context of a hyperventilation sequence, the evaluation can include assessing whether the patient's breathing is likely to be effective for provoking a hyperventilation-induced seizure. Hyperventilation effectiveness can be determined based on one or more characteristics of the patient's breathing, such as the breathing rate (e.g., breaths per minute, breath flow rate per minute) inhalation volume, exhalation volume, inhalation duration, and/or exhalation duration. The breathing characteristics can be measured using any suitable data type, such as audio data, breath flow rate data, breath volume data, facial tracking data (e.g., video data, EMG data), motion data (e.g., of the patient's face, mouth, shoulders, chest), and/or pulse oximetry data. Subsequently, the patient's breathing characteristics can be compared to one or more target breathing characteristics to determine whether the patient's hyperventilation was likely to be effective in provoking a seizure. A representative example of a method for monitoring a patient's breathing is described below in connection with FIG. 4.

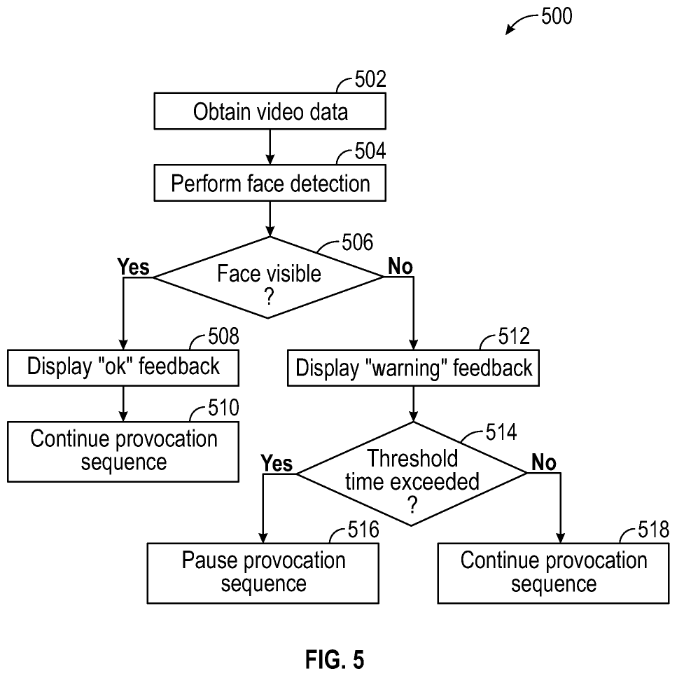

As another example, in embodiments where the patient data includes video data of the patient's eyes and/or face, the evaluation can include detecting whether the patient's eyes and/or face are sufficiently visible in the video data (e.g., centered within the field of view of the imaging device, not blurry). The detection can be performed using computer vision techniques, trained machine learning algorithms, and/or other suitable approaches known to those of skill in the art. A representative example of a method for monitoring a patient's face is described below in connection with FIG. 5.

At block 208, the method 200 can continue with outputting feedback via a user interface, based on the evaluation results. For example, if the evaluation results of block 206 indicate that the patient was not properly performing the provocation action(s), the feedback can include guidance for correcting the patient's performance. As another example, if the evaluation results of block 206 indicate that the patient data obtained during the provocation sequence was not adequately capturing the patient's state, the feedback can alert the patient that adjustments to the patient's position, sensor position, sensor state, etc., are needed. Optionally, if the evaluation results of block 206 indicates that the patient performance and patient data was satisfactory, the feedback can inform the patient that no corrective actions are needed. The feedback can be output via the user interface in various formats, such as animations, graphics, videos, text, sounds, haptics, et., as described further below.

The feedback can be presented to the patient in real-time during the provocation sequence (e.g., during the provocation period 304 of FIG. 3) so the patient can adjust their behavior and/or sensor setup, as appropriate. Subsequently, the processes of blocks 204-208 can be repeated until the provocation sequence is complete and/or sufficient patient data has been obtained.

Optionally, at block 210, the method 200 includes determining whether the patient experienced the neurological event during the provocation sequence, based on the obtained patient data (e.g., video data, audio data, electrophysiological data, motion data, and/or any of the other data types described herein). The determination can include analyzing the patient data using pattern recognition, statistical analysis, machine learning algorithms, or combinations thereof, to identify whether any neurological events occurred, and, if so, the characteristics of the events (e.g., timing, duration, severity, threshold for provocation).

For example, the process of block 210 can include detecting whether the patient successfully provoked any seizures using hyperventilation and/or other suitable techniques. In such embodiments, the process of block 210 can produce seizure data characterizing the patient's seizure threshold, such as whether any seizures were successfully provoked, and, if so, the number of seizures, the amount of time to provoke a seizure, and/or seizure duration (e.g., preictal duration, ictal duration, and/or postictal duration). In some embodiments, the seizure data includes a measurement of the patient's seizure burden during the provocation sequence, such as the frequency of seizures per unit time, the total duration of seizures per unit time, severity of loss of consciousness associated with seizures, severity of postictal state, type of seizures, seizure variability, and/or any other quantitative measure of seizure burden known those of skill in the art.

The seizure data can be determined from the patient data using any suitable technique. For example, the patient data can include eye movement data, which can be determined from video data of the patient's eyes, eye tracking data from a wearable device, etc. The seizure data can be automatically determined from the eye movement data using pattern recognition, statistical analysis, machine learning algorithms, or combinations thereof. In some embodiments, the eye movement data is converted into a time series of patient eye movements, also referred to herein as “oculometric data.” Subsequently, a seizure detection algorithm can be used to mathematically analyze the oculometric data to identify eye movement patterns that are characteristic of seizures. For example, a loss of normal eye activity (e.g., decrease in noisiness of eye movements) can correlate to the occurrence of an absence seizure. Eye movement patterns associated with seizures can be detected by changes in kurtosis, correlation, and/or other statistics calculated from eye movement data. The output of the seizure detection algorithm can be timing data (e.g., timestamps) indicating the occurrence and duration of the seizures.

In some embodiments, the seizure detection algorithm includes at least one machine learning algorithm that has been trained on previous patient data (e.g., seizure data from the same patient and/or from other patients) to identify seizures from eye movement data. The machine learning algorithm can be trained via supervised learning, unsupervised learning, semi-supervised learning, reinforcement learning, and/or transfer learning. Examples of machine learning algorithms suitable for use with the present technology include, but are not limited to, time-series algorithms (e.g., long short-term memory (LSTM), gated recurrent units (GRU)), convolutional neural networks (CNNs) (e.g., ResNet-50, GoogLeNet), and clustering algorithms (e.g., k-means clustering).

For example, the machine learning algorithm can be trained to identify seizures from the eye movement data obtained over a particular time segment. The size of the time segment can be predetermined based on the expected duration of the seizure, or can be determined dynamically based on the actual patient data. The input to the machine learning algorithm can include the eye movement time-series data, as well as features and/or statistics calculated from the time-series data (e.g., pupil size, pupil location, eccentricity, blink frequency). Optionally, the time-series data can be transformed (e.g., such as using a Fast Fourier Transform, a wavelet transform such as continuous wavelet transform (CWT)), and the transformed data (e.g., frequency domain data, scalogram data) can be used as the input to the machine learning algorithm. The input data can also include other data types, such as video data of the eyes and/or face. Based on the input data, the machine learning algorithm can determine whether a seizure occurred during the time segment, and, optionally, an associated confidence value.

In some embodiments, the seizure data is determined using other types of patient data, in addition or as an alternative to eye movement data. For example, the techniques described herein (e.g., statistical analysis, pattern recognition, machine learning) for identifying seizures can also be applied to facial movement data, brain activity data, motion data, EMG data, electrodermal data, audio data, and/or any of the other patient data types previously discussed herein.

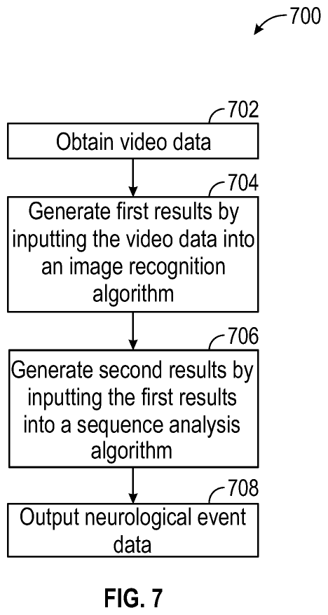

Additional examples of methods for detecting seizures are described in U.S. Patent Application Publication Nos. 2021/0000341, 2022/0022805, and 2022/0180993, the disclosures of each of which are incorporated by reference herein in their entirety. A representative example of a method for detecting a neurological event from video data is described below in connection with FIG. 7.

Optionally, the process of block 210 can also include determining whether the patient experienced any neurological events during non-provoking time periods, such as the baseline period and/or cooldown period of the provocation sequence. Patient data of all types collected during such time periods can be used in various ways, such as to calibrate and/or supplement the detection algorithms described herein, characterize the baseline and/or interictal state of the patient (e.g., whether seizures or epilepsy are present before or after provocation), and/or to describe other features of the patient data that may aid in diagnosis.

In some embodiments, the process of block 210 alternatively or additionally includes determining the patient's neurocognitive function, based on the obtained patient data. The level of neurocognitive function can be assessed based on symptoms such as drowsiness, a decrease in cognition, a decrease in attention, a decrease in concentration, a change in mood, a change in behavior, loss of consciousness, suicidality, homicidality, irritability, and/or a change in vision. Measures of neurocognitive function can be determined from eye tracking data, facial tracking data, and/or any of the other patient data types described herein using pattern recognition, statistical analysis, machine learning algorithms, or combinations thereof. For example, eye movement patterns while the patient is performing certain activities (e.g., viewing a visual stimulus, reading, focusing) can be used to characterize the patient's level of drowsiness, ability to scan natural environments, and/or reading ability, which in turn may be indicative of the patient's neurocognitive state. The patient's neurocognitive function can be used to characterize the patient's baseline and/or interictal state, assess the severity of the neurological disease or condition, identify the severity of side effects of a treatment regimen, etc. Additional examples of techniques for determining neurocognitive function from patient data are described in U.S. Patent Application Publication No. 2022/0180993, which is incorporated by reference herein in its entirety.

In some embodiments, the process of block 210 can be performed by the same device used to perform the processes of blocks 202-208. For example, the entire method 200 can be performed locally by a patient device (e.g., the patient device 104 of FIG. 1), such as a mobile device, laptop, personal computer, etc. Alternatively, the process of block 210 can be performed by a different device than the device used to perform the processes of blocks 202-208. For instance, the processes of blocks 202-208 can be performed locally by a patient device (e.g., the patient device 104 of FIG. 1), and the process of block 210 can be performed by a remote computing device or system (e.g., the system 102 of FIG. 1). In such embodiments, the patient data obtained in block 204 can be transmitted to the remote device or system via any suitable wired or wireless communication technique.

Optionally, the process of block 210 can be performed manually by a user, such as a healthcare professional (e.g., a service provider of the system 102 of FIG. 1). For example, the user can review the patient data to determine whether any neurological events occurred, and, if so, the number of events and the timing of each event (e.g., start time, end time, duration). In such embodiments, the user can be provided with tools to annotate the patient data to indicate the occurrence of neurological events. For example, the user can edit the video recording of the patient with textual and/or graphical labels, add time stamps, etc. The user can also be provided with information regarding the quality of the patient data (e.g., audio envelope, percentage of time the patient's face is visible in video data) to assess whether the patient data is suitable for use. Optionally, the user can submit a confidence rating (e.g., “not confident,” “somewhat confident,” “confident”) indicating the degree of confidence associated with each identified neurological event.

In some embodiments, the process of block 210 is performed in a semi-automated manner. For example, an automated algorithm can generate a set of initial results (e.g., candidate occurrences of neurological events), which are then sent to a user for review and/or modifications. Alternatively, a user can manually annotate or otherwise process the patient data to facilitate subsequent analysis by an automated algorithm.

The analysis results produced by the process of block 210 can be used for various aspects of patient monitoring and management, such as diagnosing the patient with the neurological disease or condition, monitoring the severity of the neurological disease or condition, assessing neurocognitive side effects, and/or evaluating the patient's response to treatment. For example, the analysis results can be transmitted to a centralized software platform (e.g., the system 102), which in turn generates a patient-specific report including information regarding the neurological events that occurred, and, optionally, the raw and/or annotated patient data obtained during the provocation attempt. The report can then be transmitted to the patient's healthcare provider (e.g., via the healthcare provider device 108) and/or to the patient (e.g., via the patient device 104) for review.

FIG. 4 is a flow diagram illustrating a method 400 for monitoring a patient's breathing, in accordance with embodiments of the present technology. The method 400 can be used to provide real-time monitoring and feedback during an attempt to provoke a seizure (or other neurological event) via hyperventilation.

The method 400 begins at block 402 with obtaining audio data of the patient's breathing during a provocation sequence. The audio data can be obtained using any suitable audio sensor, such as a built-in microphone of a patient device (e.g., a mobile device or other computing device), or a separate microphone that is operably coupled to a patient device.

At block 404, the method 400 includes determining the patient's breathing rate. The breathing rate can be determined from the audio data in many different ways. For example, the power spectrum of the audio data can be analyzed to extract the rate information. Optionally, the audio data can be filtered (e.g., using a bandpass filter and/or other technique) before the power spectrum analysis, e.g., to reduce noise. Optionally, computer vision algorithms can be used to process video data (e.g., video of the patient's mouth, face, and/or upper body) in conjunction with the audio data in order to supplement the detection of breathing rate, which may be beneficial in noisy environments and/or other situations where the audio data alone may not be sufficient for accurate detection.

At block 406, the method 400 can continue with determining whether the patient's breathing rate is within a target range for provoking a hyperventilation-induced seizure. The target range can be a predetermined range (e.g., based on clinical data, scientific literature, etc.), or can be customized to the particular patient (e.g., based on past data of the patient). For example, the target range can be from 20 breaths per minute to 24 breaths per minute.

If the patient's breathing rate is higher than the target range, the method 400 can proceed to block 408 with outputting feedback to the patient to slow down their breathing. Conversely, if the patient's breathing rate is lower than the target range, the method 400 can proceed to block 410 with outputting feedback to the patient to speed up their breathing. Optionally, if the patient's breathing rate is within the target range, the method 400 can proceed to block 412 with outputting feedback that the current breathing rate is satisfactory. The feedback can be provided in any suitable format, including graphical cues (e.g., animations corresponding to the target breathing rate and/or the patient's measured breathing rate), audio cues (e.g., a rhythmic sound corresponding to the target breathing rate), haptic cues, textual instructions (e.g., “slow down,” “speed up,” “ok”), etc.

Optionally, the method 400 can further include storing data characterizing the patient's breathing during the provocation sequence, such as the breaths per minute, breath flow rate per minute, inhalation volume, exhalation volume, inhalation duration, and/or exhalation duration. The breathing rate data can subsequently be used to evaluate the effectiveness of the hyperventilation attempt. For example, the breathing rate data can be used to generate a quality score, confidence level, or other metric indicating whether the patient's hyperventilation was likely to be effective in provoking a seizure. This information can be transmitted to a user (e.g., healthcare provider, service provider) for consideration when reviewing patient data of the provocation attempt.

The method 400 can be combined with any of the other methods described herein, such as the method 200 of FIG. 2. Additionally, the method 400 can be performed continuously during some or all of the provocation sequence 300 of FIG. 3. For instance, the method 400 can be performed repeatedly during the provocation period 304 of the provocation sequence 300 to provide continuous, real-time monitoring and feedback for the patient's breathing rate as the patient attempts to provoke a seizure. Optionally, the method 400 can also be performed during the baseline period 302 and/or the cooldown period 306 of the provocation sequence 300. In some embodiments, the method 400 includes recording the audio data only (block 402) without providing feedback to the patient during the baseline period 302 and/or cooldown period 306.

Moreover, the method 400 can be modified to incorporate other types of data for measuring the patient's breathing rate, alternatively or in addition to using audio data. For example, the patient's breathing rate can be determined using data from flow rate sensors, flow volume sensors, pressure sensors, video data, EMG data, motion data, etc.

FIG. 5 is a flow diagram illustrating a method 500 for monitoring a patient's face, in accordance with embodiments of the present technology. The method 500 can be used to provide real-time monitoring and feedback during an attempt to provoke a seizure (or other neurological event). In some embodiments, the method 500 is used in situations where video data of the patient is being recorded for use in determining whether any neurological events occurred during the provocation attempt. The method 500 can be used to monitor (1) a portion of the patient's face (e.g., upper half of the face, lower half of the face) or the entirety of the patient's face, and/or (2) one or both of the patient's eyes.

The method 500 begins at block 502 with obtaining video data of the patient during a provocation sequence. The video can be obtained using any suitable imaging device, such as a built-in camera of a patient device (e.g., a mobile device or other computing device), or a separate camera that is operably coupled to a patient device.

At block 504, the method 500 includes detecting whether the patient's face is visible in the video data. The face detection can be performed using any suitable technique, such via computer vision analysis, machine learning algorithms, and the like. For example, the patient's face can be detected using a deep learning algorithm, such as a CNN. In some embodiments, the deep learning algorithm is configured to identify a plurality of facial landmarks in order to determine which portions of the patient's face are visible in the video data. Optionally, the process of block 504 can involve outputting a confidence level for the face detection result, which may be used to assess the quality of the video data as described elsewhere herein.

At block 506, the method 500 can continue with determining whether the patient's face is visible in the video data. In some embodiments, the process of block 506 involves determining whether the entirety of the patient's face is visible. Alternatively, only certain relevant portions of the patient's face may need to be visible in the video data, such as one or both eyes, the upper half of the face, etc. If the patient's face is sufficiently visible, the method 500 can proceed to block 508 with outputting feedback that the current recording setup is satisfactory. The provocation sequence can then be continued, at block 510.

If the patient's face is not sufficiently visible, the method 500 can proceed to block 512 with outputting feedback to warning the patient to reposition their face and/or the imaging device. The feedback can be provided in any suitable format, including graphical cues (e.g., green indicator for “ok,” red indicator for warnings), audio cues, haptic cues, textual instructions (e.g., “center face”), etc.

Optionally, at block 514, the method 500 can include calculating whether the patient's face has not been sufficiently visible for a time period exceeding a threshold time. The threshold time can be any suitable time period, such as at least 1 second, 2 seconds, 3 seconds, 4 seconds, 5 seconds, 10 seconds, 15 seconds, 20 seconds, or 30 seconds. If the threshold time has not been exceeded, the provocation sequence can continue (block 518), with a warning to the patient to reposition their face. However, if the threshold time has been exceeded, the provocation sequence can be paused (block 516). For example, pausing the provocation sequence can encompass (1) pausing other types of patient monitoring (e.g., the breath rate monitoring described in connection with FIG. 4) and/or (2) pausing guidance for the provocation sequence (e.g., pausing animations, audio cues, etc., for guided hyperventilation). The provocation sequence can be resumed once the patient's face becomes sufficiently visible in the video data.

Optionally, the method 500 can further include storing data characterizing the visibility of the patient's face during the provocation sequence (e.g., the percentage of time that the patient's face was sufficiently visible). This information can subsequently be used to determine a quality score indicating whether the video data is likely to be useful in detecting neurological events. The quality score can alternatively or additionally be generated based on other factors, such as the face detection confidence level. The quality score can be transmitted to a user (e.g., healthcare provider, service provider) for consideration when reviewing patient data of the provocation attempt.

The method 500 can be combined with any of the other methods described herein, such as the method 200 of FIG. 2 and/or the method 400 of FIG. 4. Additionally, the method 500 can be performed continuously during some or all of the provocation sequence 300 of FIG. 3. For instance, the method 500 can be performed repeatedly during the baseline period 302, provocation period 304, and/or cooldown period 306 of the provocation sequence 300 to provide continuous, real-time monitoring and feedback as the patient attempts to provoke a seizure. In some embodiments, the method 500 can also be used during the baseline period 302 to ensure that the patient's face is sufficiently visible, before continuing to the provocation period 304. Additionally, although the method 500 is described above in connection with monitoring the patient's face, the method 500 can alternatively or additionally be modified for monitoring of other body parts (e.g., arms, legs, upper body, torso, the whole body).

FIGS. 6A-6D are representative examples of user interfaces (UIs) that can be displayed to a patient, in accordance with embodiments of the present technology. The UIs can be used in connection with any of the methods described herein, such as the method 200 of FIG. 2, the method 400 of FIG. 4, and/or the method 500 of FIG. 5. Additionally, the UIs can be displayed by any of the systems and devices described herein, such as the patient device 104 of FIG. 1.

FIG. 6A illustrates a UI 600a for displaying guidance for preparing for a provocation attempt, in accordance with embodiments of the present technology. In the illustrated embodiment, the UI 600a includes text and graphics instructing the patient on how to set up a mobile device (e.g., a smartphone) using a tripod accessory for recording video data of the patient's face. In other embodiments, the information displayed via the UI 600a can be modified as appropriate to provide setup instructions for other patient monitoring techniques. Optionally, the UI 600a can be used to present a tutorial (e.g., a video or animated tutorial) instructing the patient on how to perform the upcoming provocation sequence.

FIG. 6B illustrates a UI 600b for displaying guidance for positioning and recording the patient's face. The UI 600b can be displayed after the UI 600a of FIG. 6A. As shown in FIG. 6B, the UI 600b can include a video feed 602 showing the current video recorded by the camera. The UI 600b can also display a frame 604 or other suitable indicator showing where the patient's face should be positioned within the field of view of the camera. Optionally, the visual appearance of the frame 604 can be used to provide feedback on whether the patient's face is properly positioned, e.g., the frame 604 can be green if the face is sufficiently visible and/or centered, and can be red if the face is not sufficiently visible and/or off-center. Alternatively or in combination, the UI 600b can provide other types of feedback, such as audio cues, text alerts, etc.

FIG. 6C illustrates a UI 600c for displaying guidance for performing a hyperventilation sequence, in accordance with embodiments of the present technology. The UI 600c can be displayed after the UI 600b of FIG. 6B, and can be presented while the patient is attempting to provoke a seizure via hyperventilation, as described elsewhere herein. The UI 600c includes first animation 606 representing the current breathing rate of the patient. In the illustrated embodiment, for example, the first animation 606 is a pinwheel that spins in time with the patient's breathing. Alternatively, other types of graphics can be used for the first animation 606, such as a candle that is blown out when the patient exhales, a sailboat that moves when the patient exhales, a speedometer showing the breathing rate, etc. The patient may have the option to select the type of graphic for the first animation 606.

Optionally, the UI 600c can include a second animation 608 representing a target breathing rate for the hyperventilation attempt. As shown in FIG. 6C, the second animation 608 can be a metronome, clock, or other graphic whose timing matches the target breathing rate. Alternatively or in combination, the UI 600c can output a rhythmic sound (e.g., ticking, beeping) that provides an audio cue for the target breathing rate.

In some embodiments, the UI 600c also displays the video feed 602 of the patient's face, so the patient can evaluate whether their face is within the field of view of the camera. As previously described, the UI 600c can include a frame 604 that changes color and/or otherwise provides feedback if the patient's face is not sufficiently visible.

FIG. 6D illustrates a UI 600d for submitting self-reporting data, in accordance with embodiments of the present technology. The UI 600d can be displayed after the UI 600c of FIG. 6C, and can be used to provide additional data regarding the hyperventilation attempt. Alternatively or in combination, the UI 600d can be used to input other relevant patient data, such as contact information, demographic information, medication information, patient history, responses to questions, etc. For example, in some embodiments, the UI 600d can be configured to display a questionnaire (e.g., Pediatric Epilepsy Side Effects Questionnaire) to collect information regarding the patient's seizures, medications, side effects, and/or any other suitable data.

FIG. 7 is a flow diagram illustrating a method 700 for detecting a neurological event, in accordance with embodiments of the present technology. The method 700 can be used to detect the occurrence of many types of neurological events, such as seizures (e.g., absence seizures, atypical absence seizures, tonic-clonic seizures, clonic seizures, tonic seizures, atonic seizures, myoclonic seizures, simple partial seizures, complex partial seizures, secondary generalized seizures, infantile spasms), loss of consciousness and/or other states of altered consciousness (e.g., due to immediately reversible causes such as syncope and/or due to more chronic problems such as epilepsy, dementia, head injury, encephalopathy), drowsiness, etc. As described in detail below, the method 700 can utilize a two-stage machine learning algorithm to automatically identify neurological events from a video recording of the patient.

The method 700 begins at block 702 with obtaining video data of a patient. The video data can depict part or all of the patient's body (e.g., the left eye, right eye, and/or face), and can be obtained using any suitable imaging device (e.g., a camera), as described elsewhere herein. The video data can record the patient's state during a time period in which the patient is experiencing or is expected to experience the neurological event. For example, the video data can be obtained during an attempt to provoke the neurological event, e.g., as previously described in connection with FIGS. 2 and 5. The video data can be a live stream of the patient' current state, or can be a saved file that was recorded at an earlier time.

Optionally, the process of block 702 can include preparing the video data for analysis by the machine learning algorithm. This can include, for example, selecting a subset of the video frames for analysis, such as based on image quality, whether the relevant parts of the patient's body are visible, the likelihood that the neurological event occurred during those video frames (e.g., based on self-reporting, annotations by users), etc. In some embodiments, the video data is reshaped for input into the machine learning algorithm, such as by resizing the video frames to a particular size, adjusting the color of the video data (e.g., changing to grayscale or to black and white), compressing the video using an image autoencoder or other dimensionality reduction techniques, and/or any other appropriate changes to the format of the video, depending on the type of machine learning algorithm used.

At block 704, the method 700 can include generating a set of first results by inputting the video data into an image recognition algorithm. The image recognition algorithm can be a first stage of the two-stage machine learning algorithm. For example, the two-stage machine learning algorithm can be a two-stage RNN, and the first stage can be an image recognition network that has been trained to recognize and categorize objects from images. The image recognition network can include a plurality of layers, such as an input layer, an output and/or compression layer, and a plurality of hidden layers between the input layer and the output and/or compression layer. In some embodiments, the image recognition network is configured to process each video frame individually to identify and categorize relevant objects in the video frame, such as the patient's eyes, other facial features, etc. The first results output by the image recognition algorithm can be a subset of video frames with annotations labeling one or more objects of interest.

At block 706, the method 700 can continue with generating a set of second results by inputting the first results into a sequence analysis algorithm. The sequence analysis algorithm can be the second stage of the two-stage machine learning algorithm. The sequence analysis algorithm can be an algorithm that can consider information that is distributed across time, e.g., by learning how input data varies across time and/or by adjusting future predictions based on previously observed examples. Examples of sequence analysis algorithms include, but are not limited to, LSTM algorithms, CNNs, GRUs, temporal convolutional networks (TCN), attention-based neural networks (e.g., transformer networks, hierarchical attention networks), autoregressive and moving average models (e.g., ARIMA, SARIMA, and ARFIMA models), Bayesian neural networks, decision tree networks, and random convolutional kernel classification (e.g., ROCKET transforms).

For example, in embodiments where the two-stage machine learning algorithm is a two-stage RNN, the second stage can be a LSTM neural network including an input layer, an output layer, and a plurality of recurrent layers between the input and output layers. The LSTM neural network can incorporate a plurality of recurrent connections that allow the model to analyze image features that occur across multiple video frames, rather than a single video frame alone. Accordingly, the LSTM neural network can be trained to identify neurological events from video data based on changes in the patient's eyes, face, and/or other relevant anatomical features over time. In some embodiments, the LSTM operates directly on the video data itself, rather than from data (e.g., oculometric data or other time series data) extracted from the video data. The second results produced by the LSTM algorithm can be a value representing the probability that the neurological event occurred during a particular subset of video frames.

In some embodiments, the video data obtained in block 702 is the video data is divided into a plurality of shorter segments, each segment including a respective subset of video frames. Each subset of video frames can be analyzed separately by the two-stage machine learning algorithm in blocks 704 and 706 to detect whether any neurological events occurred during the corresponding video segment. The processes of blocks 704 and 706 can be repeated to sequentially analyze each segment of the video data. The results for each segment can then be combined to generate the final analysis.

At block 708, the method 700 can include outputting neurological event data, based on the second results. For example, the second results generated in block 706 can be used to determine whether any neurological events occurred in the video data, and, if so, the number of events and the timing of each event (e.g., start time, end time, duration). The neurological event data can be used to produce patient-specific reports and/or other analyses, as described elsewhere herein.

The method 700 can be combined with any of the other methods described herein, such as the method 200 of FIG. 2. For example, the method 700 can be used to analyze video data of a patient during a provocation sequence to detect whether any seizures (or other neurological events occurred). In other embodiments, however, the method 700 can be used to analyze patient data collected in other contexts, such as patient data of seizures that occurred spontaneously without voluntary provocation.

### EXAMPLES

The following examples are included to further describe some aspects of the present technology, and should not be used to limit the scope of the technology.
1. A method for monitoring a patient, the method comprising:
outputting a user interface configured to guide the patient in performing a provocation sequence for a neurological event;
obtaining patient data indicative of a state of the patient during the provocation sequence;
evaluating suitability of the patient data for detecting an occurrence of the neurological event; and
outputting feedback via the user interface based on the evaluation.
2. The method of Example 1, wherein the neurological event comprises a seizure.
3. The method of Example 2, wherein the seizure comprises an absence seizure.
4. The method of any one of Examples 1 to 3, wherein the provocation sequence is configured to induce the neurological event in the patient via hyperventilation.
5. The method of Example 4, wherein the user interface is configured to display a graphical representation of a target breathing rate for the hyperventilation.
6. The method of Example 5, wherein the graphical representation includes an animation representing the target breathing rate.
7. The method of Example 5 or 6, further comprising measuring a breathing rate of the patient, based on the patient data.
8. The method of Example 7, wherein the patient data comprises audio data, and the breathing rate is measured based on the audio data.
9. The method of Example 8, wherein the audio data is generated by a microphone of a mobile device.
10. The method of any one of Examples 7 to 9, wherein evaluating the suitability of the patient data comprises comparing the breathing rate of the patient to the target breathing rate.
11. The method of any one of Examples 7 to 10, wherein:
if the breathing rate of the patient is less than the target breathing rate, the feedback comprises instructions to increase the breathing rate, and
if the breathing rate of the patient is greater than the target breathing rate, the feedback comprises instructions to decrease the breathing rate.
12. The method of any one of Examples 7 to 11, wherein the feedback comprises an animation representing the breathing rate of the patient.
13. The method of any one of Examples 1 to 12, wherein the provocation sequence is configured to induce the neurological event in the patient via one or more of the following: photic simulation, auditory stimulation, visual patterns, reading, or concentration.
14. The method of any one of Examples 1 to 13, wherein the patient data comprises video data.
15. The method of Example 14, wherein the video data is generated by an imaging device of a mobile device.
16. The method of Example 14 or 15, wherein evaluating the suitability of the patient data comprises detecting whether the patient's face is sufficiently visible in the video data.
17. The method of Example 16, wherein, if the patient's face is not sufficiently visible in the video data, the feedback comprises instructions to reposition the patient's face.
18. The method of any one of Examples 1 to 17, wherein the provocation sequence includes a baseline period, a provocation period, and a cooldown period.
19. The method of Example 18, wherein obtaining the patient data comprises obtaining video data during the baseline period, the provocation period, and the cooldown period.
20. The method of Example 18 or 19, wherein obtaining the patient data comprises obtaining video data and audio data during the provocation period, and wherein the method further comprises:
determining that the patient's face is not sufficiently visible in the video data, and
pausing the obtaining of the audio data until the patient's face becomes sufficiently visible in the video data.
21. The method of any one of Examples 1 to 20, further comprising generating a suitability score, based on the evaluation.
22. The method of any one of Examples 1 to 21, further comprising determining whether the patient experienced the neurological event based on the patient data.
23. The method of Example 22, wherein the patient data comprises video data of the patient's face and eyes.
24. The method of Example 23, wherein determining whether the patient experienced the neurological event comprises analyzing the video data using a two-stage machine learning algorithm.
25. The method of Example 24, wherein the two-stage machine learning algorithm includes an image recognition stage and a long short-term memory stage.
26. The method of any one of Examples 1 to 25, further comprising transmitting the patient data to a remote system configured to determine whether the patient experienced the neurological event based on the patient data.
27. A device for monitoring a patient, the device comprising:
a processor;
a display operably coupled to the processor and configured to output a user interface; and
a memory operably coupled to the processor and storing instructions that, when executed by the processor, cause the device to perform operations comprising:

outputting guidance for performing a provocation sequence for a neurological event via the user interface,

obtaining patient data using at least one sensor during the provocation sequence,

determining quality of the patient data for detecting an occurrence of the neurological event, and

outputting feedback via the user interface based on the quality of the patient data.
28. The device of Example 27, wherein the neurological event comprises a seizure.
29. The device of Example 27 or 28, wherein the provocation sequence is configured to induce the neurological event in the patient via one or more of hyperventilation, photic simulation, auditory stimulation, visual patterns, reading, or concentration.
30. The device of any one of Examples 27 to 29, wherein the at least one sensor comprises an audio sensor configured to obtain audio data of the patient's breathing.
31. The device of Example 30, wherein the audio sensor is a microphone of a mobile device.
32. The device of Example 30 or 31, wherein the operations further comprise measuring a breathing rate of the patient, based on the audio data.
33. The device of Example 32, wherein the feedback comprises a graphical representation of the breathing rate.
34. The device of Example 32 or 33, wherein determining the quality of the patient data comprises comparing the breathing rate of the patient to a target breathing rate.
35. The device of Example 34, wherein:
if the breathing rate of the patient is less than the target breathing rate, the feedback comprises instructions to increase the breathing rate, and
if the breathing rate of the patient is greater than the target breathing rate, the feedback comprises instructions to decrease the breathing rate.
36. The device of any one of Examples 32 to 35, wherein the user interface is configured to display a graphical representation of the target breathing rate.
37. The device of any one of Examples 27 to 36, wherein the at least one sensor comprises an imaging device configured to obtain video data of the patient's face.
38. The device of Example 37, wherein the imaging device is a camera of a mobile device.
39. The device of Example 37 or 38, wherein determining the quality of the patient data comprises detecting whether the patient's face is sufficiently visible in the video data.
40. The device of Example 39, wherein, if the patient's face is not sufficiently visible in the video data, the feedback comprises instructions to reposition the patient's face.
41. The device of any one of Examples 27 to 40, further comprising the at least one sensor.
42. The device of any one of Examples 27 to 41, wherein the processor is configured to receive the patient data from a second device, and the second device includes the at least one sensor.
43. The device of Example 42, wherein the second device comprises an eye tracking device.
44. The device of Example 42 or 43, wherein the second device comprises a facial tracking device.
45. The device of any one of Examples 42 to 44, wherein the second device is a wearable device.
46. The device of any one of Examples 27 to 45, wherein the device is a mobile device.
47. The device of any one of Examples 27 to 46, wherein the provocation sequence includes a baseline period, a provocation period, and a cooldown period.
48. The device of any one of Examples 27 to 47, wherein the operations further comprise determining whether the patient experienced the neurological event based on the patient data.
49. The device of Example 48, wherein determining whether the patient experienced the neurological event comprises analyzing the video data using a two-stage machine learning algorithm.
50. The device of any one of Examples 27 to 49, wherein the operations further comprise transmitting the patient data to a remote server configured to determine whether the patient experienced the neurological event based on the patient data.
51. A non-transitory computer-readable storage medium comprising instructions that, when executed by one or more processors of a computing system, cause the computing system to perform operations comprising:
outputting visual guidance configured to assist a patient in performing a provocation sequence for a neurological event;
obtaining patient data indicative of a state of the patient during the provocation sequence;
evaluating suitability of the patient data for detecting an occurrence of the neurological event; and
outputting feedback to the patient based on the evaluation.

### Conclusion

Although many of the embodiments are described above with respect to systems, devices, and methods for monitoring and managing epilepsy, the technology is applicable to other applications and/or other approaches, such as monitoring and managing other types of neurological diseases and conditions that are associated with loss of consciousness and/or lapses in cognitive function, as well as neurodegenerative diseases. Examples of such diseases and conditions include, but are not limited to, Tay-Sachs disease, ADHD, Parkinson's disease, syncope, and rare/orphan diseases such as tuberous sclerosis and Rett syndrome. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-7.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The embodiments described above may be implemented in many different ways. In some embodiments, the various computing systems described herein may each be implemented by a separate or shared physical or virtual general-purpose computer having one or more central processor(s), memor(ies), disk or other mass storage device(s), communication interface(s), input/output (I/O) device(s), and other peripherals. The processors may operate, for example, by loading software instructions, and then executing the instructions to carry out the functions described. As is known in the art, such a computer may contain a system bus, where a bus is a set of hardware wired connections used for data transfer among the components of a computer or processing system. The bus or busses are shared conduit(s) that connect different elements of the computer system (e.g., processor, disk storage, volatile and non-volatile memory, input/output ports, network ports, etc.) to enable the transfer of information. One or more central processor units are attached to the system bus and provide for the execution of computer instructions. Also attached to the system bus are typically I/O device interfaces for connecting various input and output devices (e.g., keyboard, mouse, displays, printers, speakers, etc.) to the computer. Network interface(s) allow the computer to connect to various other devices attached to a network. Memory provides volatile or non-volatile storage for computer software instructions and data used to implement an embodiment. Disk or other mass storage provides non-volatile storage for computer software instructions and data used to implement, for example, the various procedures described herein.

Embodiments may therefore typically be implemented in hardware, firmware, software, or any combination thereof. In some embodiments, the computers that execute the processes described above are deployed in a cloud computing arrangement that makes available one or more physical and/or virtual data processing machines via a convenient, on-demand network access model to a shared pool of configurable computing resources (e.g., networks, servers, storage, applications, and services) that can be rapidly provisioned and released with minimal management effort or service provider interaction. Such cloud computing deployments can allow multiple users to access computing resources. By aggregating demand from multiple users in central locations, cloud computing environments can be built in data centers that use the best and newest technology, located in the sustainable and/or centralized locations and designed to achieve the greatest per-unit efficiency possible.

Furthermore, firmware, software, routines, or instructions may be described herein as performing certain actions and/or functions. It also should be understood that the block and network diagrams may include more or fewer elements, be arranged differently, or be represented differently. Therefore, it will be appreciated that such descriptions contained herein are merely for convenience and that such actions can result from computing devices, processors, controllers, or other devices executing the firmware, software, routines, instructions, etc.

Other modifications and variations are possible in light of the above teachings. For example, while a series of steps has been described above with respect to the flow diagrams, the order of the steps may be modified in other embodiments. In addition, the steps and operations may be performed by additional or other modules or entities, which may be combined or separated to form other modules or entities. For example, while a series of steps has been described with regard to certain figures, the order of the steps may be modified in other embodiments consistent with the principles of the present technology. Further, non-dependent steps may be performed in parallel. Further, disclosed embodiments may not be limited to any specific combination of hardware.

Certain portions may be implemented as “logic” that performs one or more functions. This logic may include hardware, such as hardwired logic, an application-specific integrated circuit, a field programmable gate array, a microprocessor, software, firmware, or a combination thereof. Some or all of the logic may be stored in one or more tangible non-transitory computer-readable storage media and may include computer-executable instructions that may be executed by a computer or data processing system. The computer-executable instructions may include instructions that implement one or more embodiments described herein. The tangible non-transitory computer-readable storage media may be volatile or non-volatile and may include, for example, flash memories, dynamic memories, removable disks, and non-removable disks.

Accordingly, further embodiments may also be implemented in a variety of computer architectures, physical, virtual, cloud computers, and/or some combination thereof, and thus the computer systems described herein are intended for purposes of illustration only and not as a limitation of the embodiments.

In practicing the subject methods, determining the presence or absence of a change in a signal may involve machine learning. Machine learning techniques and computational methods may be used for predicting seizures, syncope, drowsiness, loss of consciousness, and/or other neurological events or conditions from the data obtained. The machine learning process may involve relating the numerical data to the outcomes, which applies categorical training to detect and/or predict a condition or event. In some embodiments, machine learning models may include aspects of signal acquisition, signal pre-processing, features extraction from the signals, and classification between different seizure states. The disclosed methods and systems may also include confirming the presence or absence of a change relative to baseline, performing lower order statistical analysis and/or a higher order statistical analysis of the data.

Open-source tools may be employed to develop the methods described herein. This may include numerical processing languages such as Python or R, and deep learning development toolkits, such as TensorFlow, PyTorch, and Keras, to name a few. Commercially available tools such as MATLAB's Statistics and Machine Learning Toolbox™, Neural Network Toolbox™, Image Processing Toolbox™, the Image Acquisition Toolbox™, Mapping Toolbox™ and other MATLAB tools, such as the MATLAB Signal Processing Toolbox™ may also be leveraged to provide the machine learning and signal processing methods described herein.

No element, act, or instruction used herein should be construed as critical or essential to the disclosure unless explicitly described as such. Also, as used herein, the article “a” is intended to include one or more items. Where only one item is intended, the term “one” or similar language is used. Further, the phrase “based on” is intended to mean “based, at least in part, on” unless explicitly stated otherwise.

Also, the term “user,” as used herein, is intended to be broadly interpreted to include, for example, a computer or data processing system or a human user of a computer or data processing system, unless otherwise stated.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method for monitoring a patient, the method comprising:
outputting a user interface configured to guide the patient in performing a provocation sequence for a neurological event;
obtaining patient data indicative of a state of the patient during the provocation sequence;
evaluating suitability of the patient data for detecting an occurrence of the neurological event; and
outputting feedback via the user interface based on the evaluation.

2. The method of claim 1, wherein the neurological event comprises a seizure.

3. The method of claim 2, wherein the seizure comprises an absence seizure.

4. The method of claim 1, wherein the provocation sequence is configured to induce the neurological event in the patient via hyperventilation.

5. The method of claim 4, wherein the user interface is configured to display a graphical representation of a target breathing rate for the hyperventilation.

6. The method of claim 5, wherein the graphical representation includes an animation representing the target breathing rate.

7. The method of claim 5, further comprising measuring a breathing rate of the patient, based on the patient data.

8. The method of claim 7, wherein the patient data comprises audio data, and the breathing rate is measured based on the audio data.

9. The method of claim 8, wherein the audio data is generated by a microphone of a mobile device.

10. The method of claim 7, wherein evaluating the suitability of the patient data comprises comparing the breathing rate of the patient to the target breathing rate.

11. The method of claim 10, wherein:
if the breathing rate of the patient is less than the target breathing rate, the feedback comprises instructions to increase the breathing rate, and
if the breathing rate of the patient is greater than the target breathing rate, the feedback comprises instructions to decrease the breathing rate.

12. The method of claim 7, wherein the feedback comprises an animation representing the breathing rate of the patient.

13. The method of claim 1, wherein the patient data comprises video data.

14. The method of claim 13, wherein the video data is generated by an imaging device of a mobile device.

15. The method of claim 13, wherein evaluating the suitability of the patient data comprises detecting whether the patient's face is sufficiently visible in the video data.

16. The method of claim 15, wherein, if the patient's face is not sufficiently visible in the video data, the feedback comprises instructions to reposition the patient's face.

17. The method of claim 1, wherein the provocation sequence includes a baseline period, a provocation period, and a cooldown period.

18. The method of claim 1, further comprising determining whether the patient experienced the neurological event based on the patient data.

19. The method of claim 18, wherein the patient data comprises video data of the patient's face and eyes, and wherein determining whether the patient experienced the neurological event comprises analyzing the video data using a two-stage machine learning algorithm.

20. The method of claim 1, further comprising transmitting the patient data to a remote system configured to determine whether the patient experienced the neurological event based on the patient data.
